(19) United States
(12) Patent Application Publication
(10) Pub. No.: US 2024/0382407 A1
(43) Pub. Date: Nov. 21, 2024

(54) PROCESS FOR TREATING KERATINOUS MATERIAL, COMPRISING THE MIXING OF AT LEAST TWO AGENTS (A) AND (B)

(71) Applicant: Henkel AG & Co. KGaA, Duesseldorf (DE)

(72) Inventors: Constanze Kruck, Grevenbroich (DE); Claudia Kolonko, Remscheid (DE); Marcus Claas, Hilden (DE); Gabriele Weser, Essen (DE)

(21) Appl. No.: 18/786,168

(22) Filed: Jul. 26, 2024

Related U.S. Application Data
(63) Continuation of application No. PCT/EP2022/085851, filed on Dec. 14, 2022.

(30) Foreign Application Priority Data
Jan. 26, 2022 (DE) 102022200866.9

(51) Int. Cl. A61K 8/898; A61Q 5/02; A61Q 5/10

(52) U.S. Cl. CPC A61K 8/898; A61Q 5/02; A61Q 5/10; A61K 2800/43; A61K 2800/54; A61K 2800/884

(57) ABSTRACT

A method for coloring keratin fibers, in particular human hair, including applying a coloring agent (F) to the keratin fibers and applying a shampoo (S) to the keratin fibers. The coloring agent includes at least one amino-functionalized silicone polymer, and at least one pigment, and the shampoo includes at least one amino-functionalized silicone polymer. The coloring agent (F) and the shampoo (S) are different formulations.

### FIELD OF INVENTION

The present application relates to a method for coloring keratin fibers, in particular human hair, which method comprises the application of a coloring agent (F) and the application of a shampoo (S). In this case, the coloring agent (F) contains at least one amino-functionalized silicone polymer (f1), and at least one pigment (f2). The shampoo (S) contains at least one amino-functionalized silicone polymer (s1).

The present application relates secondly to a multi-component packaging unit (kit-of-parts) which contains the above-described coloring agent (F) and the shampoo (S) in separately packaged containers.

This application thirdly relates to the use of the above-described shampoo (S) for improving color retention on keratin fibers which have been colored by applying a coloring agent (F) containing at least one amino-functionalized silicone polymer (f1) and at least one pigment (f2).

### BACKGROUND

Changing the shape and color of keratin fibers, in particular human hair, is an important field of modern cosmetics. To change the hair color, the skilled artisan is familiar with a variety of coloring systems depending on the coloring requirements. Oxidation coloring agents are typically used for permanent, intense coloring with good fastness properties and good gray coverage. Such coloring agents contain oxidation dye precursors, what are known as developer components and coupler components, which together form the actual dyes under the influence of oxidizing agents such as, for example, hydrogen peroxide. Oxidation coloring agents are characterized by very long-lasting color results.

When using direct dyes, dyes which are already formed diffuse out of the coloring agent into the hair fiber. In comparison with oxidative hair coloring, the colors obtained with direct dyes have a lower durability and a more rapid washing out. Colors with direct dyes usually remain on the hair for a period of between 5 and 20 hair washes.

### SUMMARY OF INVENTION

The use of color pigments for brief changes in color on the hair and/or the skin is known. Pigments or color pigments are generally understood to mean insoluble coloring substances. These are present undissolved in the form of small particles in the coloring formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting coloring, the use of oxidative coloring agents has hitherto been the only option. However, despite multiple optimization attempts, an unpleasant ammonia odor or amine odor cannot be completely avoided in oxidative hair coloring. The hair damage that remains associated with the use of the oxidative coloring agents also has a disadvantageous effect on the hair of the user. The search for alternative, high-performance coloring methods is therefore an ongoing challenge. One possible alternative coloring system, which recently has been moving increasingly into focus, is based on the use of colored pigments.

Coloring with pigments offers various major advantages. Since the pigments are deposited only from the outside to the keratin materials, in particular to the hair fibers, the damage associated with the coloring process is very particularly low. Furthermore, coloring that is no longer desired can be removed quickly and easily without residue and therefore offers the user the possibility of returning directly and without great effort to their original hair color. This coloring process is therefore particularly attractive for consumers who do not regularly want to re-color their hair.

In current works, the problem of the low durability of this coloring system has been addressed. In this context, it was found that the wash fastness of the coloring results obtained with pigments could be greatly improved by combining the pigments with certain amino-functionalized silicone polymers. Despite the options thus found for improving wash fastness and color retention, there is still a need for optimization in this context. For this reason, options are still being sought to further improve a pigment-based coloring system in terms of its wash fastness and color retention.

The object of the present invention is that of providing a coloring system which, if possible, has fastness properties comparable to oxidative coloring. In particular, the wash-fastness properties should be outstanding, but the use of the oxidation dye precursors normally used for this purpose should be avoided. A technique has been sought that makes it possible to fix the coloring compounds (in particular pigments) known from the prior art, in an extremely durable manner, on the hair. When using the agent in a coloring method, intense coloring results with good fastness properties and good color retention should be achieved.

Surprisingly, it has now been found that keratin fibers that have been colored in a first step by applying a coloring agent having an aminosilicone and pigment have an improved wash fastness, particularly when a shampoo that likewise contains an aminosilicone is applied in a subsequent step.

### DETAILED DESCRIPTION OF THE INVENTION

The present invention firstly relates to a method for coloring keratin fibers, in particular human hair, comprising
(1) applying a coloring agent (F) to the keratin fibers which contains
(f1) at least one amino-functionalized silicone polymer, and
(f2) at least one pigment, and
(2) applying a coloring agent (F) to the keratin fibers which contains
(s1) at least one amino-functionalized silicone polymer,
wherein the coloring agent (F) and the shampoo (S) are different formulations.

The work leading to this invention has shown that keratin fibers, in particular hair, could be intensely colored by applying coloring agents containing the aminosilicone (f1) and pigment (f2). The wash fastnesses of the colorings obtained in this manner could be further improved if a shampoo (S) which also contained an amino-functionalized silicone polymer (s1) was applied at least once to the keratin fibers after the coloring. Without being limited to this theory, it is believed that the post-coloring application of the shampoo (S) reinforces the film of silicone and pigment formed in the coloring process or closes gaps in the film due to the presence of the aminosilicone (s1) in the shampoo. In this way, the deterioration or damage to the film that inevitably occurs due to contact with the surfactants present in a shampoo can be compensated for.

### Keratin Fibers

Keratin fibers are understood to be wool, fur and feathers, and in particular human hair. Keratin fibers are very particularly preferably understood to mean human hair.

### Coloring Agent

Within the scope of this invention, the term “coloring agent” is used for a coloring of the keratin material, in particular hair, brought about by use of pigments. In this coloring process, the pigments are deposited as coloring compounds together with the amino-functionalized silicone polymer(s) in a particularly homogeneous, uniform and smooth film on the surface of the keratin material.

### Application of the Coloring Agent (F) to Keratin Fibers

In step (1) of the coloring method according to the invention, a coloring agent (F) is applied to the keratin fibers or to the human hair which contains at least one amino-functionalized silicone polymer (f1) and at least one pigment (f2). The coloring agent (F) is a ready-to-use coloring agent.

### Amino-Functionalized Silicone Polymer (f1) in the Coloring Agent

The coloring agent (F) contains at least one amino-functionalized silicone polymer (f1). The amino-functionalized silicone polymer can alternatively also be referred to as aminosilicone or amodimethicone.

Silicone polymers are generally macromolecules with a molecular weight of at least 500 g/mol, preferably at least 1000 g/mol, more preferably at least 2500 g/mol and more preferably at least 5000 g/mol which comprise repeating organic units.

The maximum molecular weight of the silicone polymer depends on the degree of polymerization (number of polymerized monomers) and the batch size, and is also determined by the polymerization method. In the context of the present invention, it is preferable if the maximum molecular weight of the silicone polymers is not more than 107 g/mol, preferably not more than 106 g/mol, and particularly preferably not more than 10 g/mol.

The silicone polymers comprise many Si—O repeat units, the Si atoms being able to bear organic groups such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units and particularly preferably more than 100 Si—O repeat units, very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group and a tertiary amino group. All these amino groups can be protonated in an acidic environment and are then present in their cationic form.

In principle, a good coloring performance with amino-functionalized silicone polymers were achieved if these bear at least one primary, at least one secondary and/or at least one tertiary amino group. However, intense colorings with the best colorfastness were obtained when an amino-functionalized silicone polymer containing at least one secondary amino group had been used in the agent.

In a very particularly preferred embodiment, a method according to the invention is characterized in that a coloring agent (F) is applied to the keratin fibers which comprises at least one amino-functionalized silicone polymer (f1) having at least one secondary amino group.

The secondary amino groups(s) may be at different positions of the amino-functionalized silicone polymer. Very particularly good color results were obtained when an amino-functionalized silicone polymer had been used that had at least one, preferably several structural units of the formula (Si-amino).

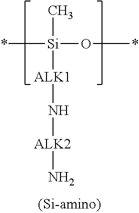

In the structural units of the formula (Si-amino), the abbreviations ALK1 and ALK2 are each independently a linear or branched divalent C₁-C₂₀ alkylene group.

In a further very particularly preferred embodiment, a method according to the invention is characterized in that the coloring agent (F) contains at least one amino-functionalized silicone polymer (f1) which comprises at least one structural unit of formula (Si-amino),

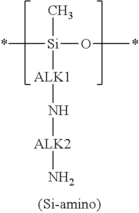

where
ALK1 and ALK2 represent, independently of one another, a linear or branched divalent C₁-C₂₀ alkylene group.

The positions marked with an asterisk (*) in each case indicate the bond to further structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—) and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀-alkylene groups are (—CH₂—CH(CH₃)—) and (—CH₂—CH(CH₃)—CH₂—).

In a further particularly preferred embodiment, the structural units of the formula (Si-amino) represent repeat units in the amino-functionalized silicone polymer, so that the silicone polymer comprises a plurality of structural units of the formula (Si-amino).

In the following, particularly well-suited amino-functionalized silicone polymers with at least one secondary amino group are listed.

Colorings with the very best wash fastness could be obtained if at least one agent containing at least one amino-functionalized silicone polymer comprising structural units of formula (Si-I) and formula (Si-II) was applied to the keratin material during the coloring.

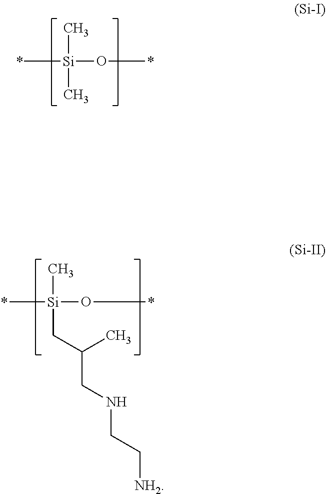

In a further explicitly very particularly preferred embodiment, a method according to the invention is characterized in that the coloring agent (F) contains at least one amino-functionalized silicone polymer (a1) which comprises structural units of formula (Si-I) and formula (Si-II):

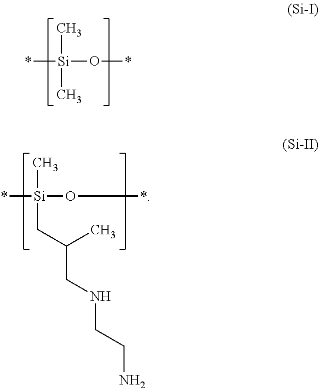

A corresponding amino-functionalized silicone polymer with the structural units (Si-I) and (Si-II) is, for example, the commercial product DC 2-8566 or Dowsil 2-8566 Amino Fluid which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. A further particularly preferred commercial product is Dowsil AP-8568 Amino Fluid, which is likewise sold commercially by Dow Chemical Company.

In the context of a further embodiment, the coloring can also be carried out by applying a coloring agent (F) which contains at least one amino-functional silicone polymer of formula (Si-III),

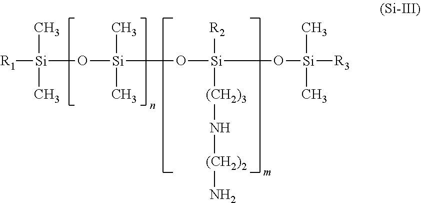

where
   m and n denote numbers that are selected such that the sum (n+m) is in a range of 1 to 1000,
   n is a number in a range of 0 to 999 and m is a number in a range of 1 to 1000,
   R1, R2 and R3, which are identical or different, denote a hydroxyl group or a C1-4 alkoxy group,
   at least one of the R1 to R3 groups denoting a hydroxyl group.

Further suitable methods are characterized by the of a coloring agent to the keratin fibers, wherein the coloring agent contains at least amino-functional silicone polymer of formula (Si-IV),

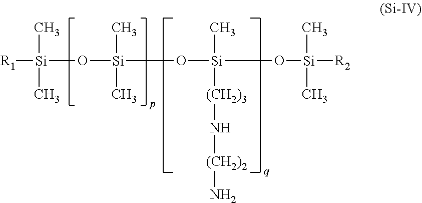

where
   p and q denote numbers that are selected such that the sum (p+q) is in a range of 1 to 1000,
   p is a number in a range of 0 to 999 and q is a number in a range of 1 to 1000, and
   R1 and R2, which are different, denote a hydroxyl group or a C1-4 alkoxy group, at least one of the groups R1 to R2 denoting a hydroxyl group.

The silicones of the formulas (Si-III) and (Si-IV) differ by the grouping on the Si atom that carries the nitrogen-containing group: In formula (Si-III), R2 denotes a hydroxyl group or a C1-4 alkoxy group, whereas the group in formula (Si-IV) is a methyl group. The individual Si moieties, which are labeled with the indices m and n or p and q, need not be present as blocks; instead, the individual units can also be distributed randomly, i.e., in the formulas (Si-III) and (Si-IV), each R1-Si(CH₃)₂ group is not necessarily bound to a —[O—Si(CH₃)₂] moiety.

Methods according to the invention in which a coloring agent containing at least one amino-functional silicone polymer of formula (Si—V) is applied to the keratin fibers have also proven to be particularly effective with regard to the generation of intense color results

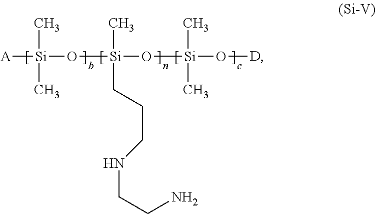

where
A represents an —OH, —O—Si(CH₃)₃, —O—Si(CH₃)₂OH, or —O—Si(CH₃)₂OCH₃ group
   D represents an —H, —Si(CH₃)₃, —Si(CH₃)₂OH, or —Si(CH₃)₂OCH₃ group,
b, n and c stand for integers between 0 and 1000, with the proviso that
   n>0 and b+c>0
   at least one of the conditions A=—OH or D=—H is met.

In the aforementioned formula (Si—V), the individual siloxane units having the indices b, c, and n are randomly distributed, i.e., they are not necessarily block copolymers.

The applied coloring agent can also contain one or more different amino-functionalized silicone polymers which are described by the formula (Si—VI):

M(RₐQ_{b}SiO_{(4-a-b)/2)x}(R_{c}SiO_{(4-c)/2)y}M (Si—VI).

In the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, this structure comprising diamino ring structures, such as piperazinyl. Z is most preferably a —NHCH₂CH₂NH₂ group. Another possible formula for Z is N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units lies within a range of about 1:2 to 1:65, preferably about 1:5 to about 1:65 and most preferably about 1:15 to about 1:20. If one or more silicones of the above formula are used, then the various variable substituents in the above formula can be different in the various silicone components, present in the silicone mixture.

In the context of a further embodiment, a method according to the invention is characterized by the application of a coloring agent to the keratin fibers, wherein the coloring agent is an amino-functional silicone polymer of formula (Si—VII)

R′ₐG₃₋ₐ-Si(OSiG₂)ₙ-(OSiG_{b}R′_{2-b})ₘ—O-SiG₃₋ₐ-R′ₐ (Si—VII),
in which:
   G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
   a represents a number between 0 and 3, in particular 0;
   b represents a number between 0 and 1, in particular 1;
   m and n are numbers whose sum (m+n) is between 1 and 2000, preferably between 50 and 150, n preferably assuming values of 0 to 1999 and in particular from 49 to 149, and m preferably assuming values of 1 to 2000, in particular from 1 to 10;
   R′ is a monovalent group selected from:
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻
each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—,
R″ representing identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion preferably selected from chloride, bromide, iodide or methosulfate.

In the context of a further embodiment, a method according to the invention is characterized by the application of a coloring agent to the keratin fibers, wherein the coloring agent contains at least one amino-functional silicone polymer of formula (Si-VIIa),

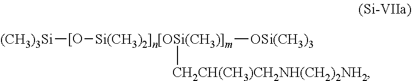

in which m and n are numbers of which the sum (m+n) is between 1 and 2000, preferably between 50 and 150, n preferably assuming values of 0 to 1999 and in particular from 49 to 149 and m preferably assuming values of 1 to 2000, in particular from 1 to 10.

These silicones are designated as trimethylsilylamodimethicones in accordance with the INCI Declaration.

In the context of a further preferred embodiment, a method according to the invention is characterized by the application of a coloring agent to the keratin fibers, wherein the coloring agent contains at least one amino-functional silicone polymer of formula (Si-VIIb),

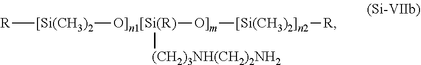

in which R denotes —OH, —O—CH₃, or a —CH₃ group and m, n1 and n2 are numbers whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

According to the INCI Declaration, these amino-functionalized silicone polymers are referred to as amodimethicones.

Irrespective of which amino-functional silicones are used, coloring agents according to the invention that contain an amino-functional silicone whose amine value is above 0.25 meq/g, preferably above 0.3 meq/g, and in particular above 0.4 meq/g are preferred. The amine value here represents the milliequivalents of amine per gram of the amino-functional silicone. Said value can be determined by titration and may also be given in the unit mg KOH/g.

Furthermore, coloring agents which contained a specific 4-morpholinomethyl-substituted silicone polymer are also suitable for use in the method according to the invention. This amino-functionalized silicone polymer comprises structural units of formulas (Si—VIII) and of the formula (Si-IX):

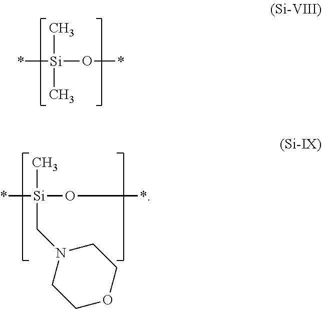

Corresponding 4-morpholinomethyl-substituted silicone polymers are described below.

A corresponding amino-functionalized silicone polymer is known by the name
amodimethicone/morpholinomethyl silsesquioxane copolymer and is commercially available in the form of the raw material Belsil ADM 8301 E from Wacker.

For example, a silicone which has structural units of formulas (Si—VIII), (Si-IX) and (Si—X) can be used as 4-morpholinomethyl-substituted silicone:

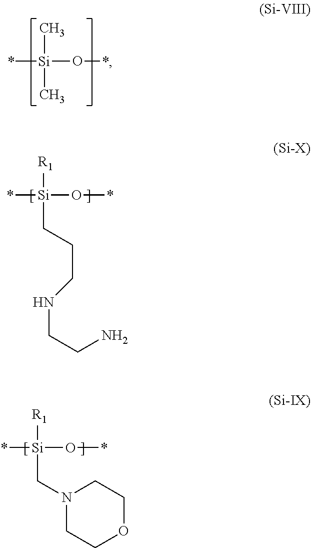

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred coloring agents contain at least one 4-morpholinomethyl-substituted silicone of formula (Si—XI)

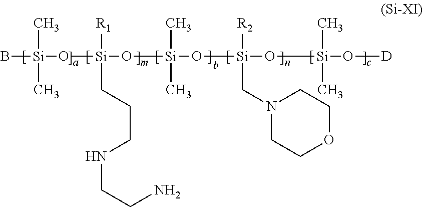

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b and c represent, independently of one another, integers between 0 and 1000, with the proviso that a+b+c>0
m and n represent, independently of one another, integers between 1 and 1000,
with the proviso that
   at least one of the conditions B=—OH or D=—H is met,
   the units a, b, c, m and n are distributed randomly or in blocks in the molecule.

Structural formula (Si—XI) is intended to indicate that the siloxane groups n and m do not necessarily have to be directly bonded to an end group B or D. Instead, in preferred formulas (Si—VI), a>0 or b>0 and, in particularly preferred formulas (Si—VI), a>0 and c>0; i.e., the terminal group B or D is preferably bonded to a dimethylsiloxy group. In formula (Si—VI) as well, the siloxane units a, b, c, m and n are preferably distributed randomly.

The silicones represented by formula (Si—VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.

These silicones lead to enormous improvements in the hair properties of hair treated with the agents according to the invention, and to greatly improved protection during oxidative treatment.

The coloring agents used in the coloring step can contain one or more amino-functionalized silicone polymers for example in a total amount of from 0.1 to 8.0 wt. %, preferably from 0.2 to 5.0 wt. %, more preferably from 0.3 to 3.0 wt. % and very particularly preferably from 0.4 to 2.5 wt. %. Here, the indicated amounts relate to the total amount of all aminosilicones used, which is set in relation to the total weight of the coloring agent.

In the context of another particularly preferred embodiment, a method according to the present invention is characterized in that the coloring agent (F)—based on the total weight of the coloring agent (F)—contains one or more amino-functionalized silicone polymers (f1) in a total amount of 0.1 to 20 wt. %, preferably from 0.2 to 10 wt. %, more preferably from 0.3 to 5 wt. %, still more preferably from 0.4 to 3.5 wt. %, and very particularly preferably from 0.5 to 2.0 wt. %.

### Pigments (f2) in the Coloring Agent (F)

As a second essential component, the coloring agent (F) applied in step (1) of the method according to the invention contains at least one pigment (f2).

Pigments within the meaning of the present invention are understood to mean coloring compounds which have a solubility of less than 0.5 g/L, preferably of less than 0.1 g/L, even more preferably of less than 0.05 g/L, at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable dye pigments may be of inorganic and/or organic origin. In a preferred embodiment, a method according to the invention is characterized in that the post-treatment agent is applied to keratin material which has been dyed by application of at least one inorganic and/or organic pigment.

Preferred color pigments are selected from synthetic or natural inorganic pigments. Inorganic color pigments of natural origin can be produced, for example, from chalk, ocher, umbra, green soil, burnt Sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, Cl 77007, Pigment Blue 29), chromium oxide hydrate (CI77289), Iron Blue (ferric ferrocyanide, CI77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, a method according to the invention is characterized in that the coloring agent (F) contains at least one inorganic pigment (a2), wherein the inorganic pigment is preferably selected from the group consisting of colored metal oxides, metal hydroxides, metal oxide hydrates, silicates, metal sulfides, complex metal cyanides, metal sulphates, bronze pigments and/or from mica-based colored pigments which are coated with at least one metal oxide and/or a metal oxychloride.

In another preferred embodiment, a method according to the invention is characterized in that the coloring agent (F) contains at least one pigment (f2) selected from mica-based colored pigments which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288) and/or iron blue (ferric ferrocyanide, CI 77510).

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)

Additional particularly preferred color pigments with the trade name
Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.
In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In the context of a further embodiment, the applied coloring agent can also contain one or more organic pigments.

The organic pigments according to the invention are correspondingly insoluble, organic dyes or color lakes, which may be selected, for example, from the group of nitroso, nitro, azo, xanthene, anthraquinone, isoindolinone, isoindoline, quinacridone, perinone, perylene, diketopyrrolopyrrole, indigo, thioindigo, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a method according to the invention is characterized in that the coloring agent (F) contains at least one organic pigment (f2), wherein the organic pigment is preferably selected from the group consisting of carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lake. The term color lake within the meaning of the invention is understood to mean particles which comprise a layer of absorbed dyes, with the unit consisting of particles and dye being insoluble under the above-mentioned conditions. The particles may be, for example, inorganic substrates which may be aluminum, silica, calcium borosilicate, calcium aluminum borosilicate or aluminum.

For example, the alizarin color lake can be used as the color lake.

For the coloring of the keratin fibers, pigments of a specific shape may also be used. For example, a pigment based on a lamellar and/or lenticular small substrate plate may be used. Furthermore, the coloring is also possible based on a small substrate plate which comprises a vacuum-metalized pigment.

In the context of another preferred embodiment, a method according to the invention is characterized in that the coloring agent (F) contains at least one pigment (f2) selected from the group of pigments based on a lamellar substrate plate, pigments based on a lenticular substrate plate, and vacuum-metalized pigments.

The small substrate plates of this type have an average thickness of at most 50 nm, preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each substrate plate has as uniform a thickness as possible. Due to the small thickness of the substrate plates, the pigment has a particularly high covering power.

The substrate plates have a preferably monolithic structure. Monolithic in this context means consisting of a single self-contained unit without fractures, stratifications or inclusions, although structural changes may, however, occur within the substrate plates. The substrate plates are preferably composed homogeneously, i.e., there is no concentration gradient within the plates. In particular, the small substrate plates are not constructed in layers and have no particles distributed therein.

The size of the substrate plate can be matched to the respective application, in particular to the desired effect on the keratin material. In general, the small substrate plates have an average largest diameter of approximately 2 to 200 μm, in particular approximately 5 to 100 μm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Helos type, having QUIXEL wet dispersion. To prepare the sample, the sample to be investigated was pre-dispersed in isopropanol for a period of 3 minutes.

The small substrate plates may be constructed from any material that can be made into the form of a small plate.

They can be of natural origin, but can also be produced synthetically. Materials from which the small substrate plates can be constructed are, for example, metals and metal alloys, metal oxides, preferably aluminum oxide, inorganic compounds, and minerals such as mica and (semi-)precious stones, as well as plastics materials. Preferably, the small substrate plate are made of metal (alloy)s.

Any metal suitable for metallic luster pigments is suitable as the metal. Such metals are, inter alia, iron and steel, and all air-resistant and water-resistant (semi) metals such as, for example, platinum, zinc, chromium, molybdenum and silicon, as well as alloys thereof such as aluminum bronzes and brass. Preferred metals are aluminum, copper, silver and gold. Preferred small substrate plates are small aluminum plates and small brass plates, small substrate plates made of aluminum being particularly preferred.

Lamellar small substrate plates are characterized by an irregularly structured edge, and are also referred to as “cornflakes” due to their appearance.

Due to their irregular structure, pigments based on lamellar small substrate plates produce a high fraction of scattered light. In addition, the pigments based on lamellar small substrate plates do not completely cover the existing color of a keratin material and, for example, effects can be achieved analogously to a natural graying.

Lenticular (=lens-shaped) small substrate plates have a substantially regular round edge and are also referred to as “silver dollars” due to their appearance. Due to their regular structure, the fraction of the reflected light predominates in the case of pigments based on lenticular small substrate plates.

Vacuum metalized pigments (VMP) can be obtained, for example, by releasing metals, metal alloys or metal oxides from correspondingly coated films. These are characterized by a particularly small thickness of the small substrate plates in the range from 5 to 50 nm and by a particularly smooth surface having increased reflectivity. Substrate plates which comprise a pigment metalized in a vacuum are also referred to as VMP substrate plates in the context of this application. VMP substrate plates of aluminum can be obtained, for example, by releasing aluminum from metalized films.

The small substrate plates made of metal or metal alloy can be passivated, for example by anodizing (oxide layer) or chromatizing.

Uncoated lamellar, lenticular and/or VPM substrate platelets, in particular those made of metal or metal alloy, reflect the incident light to a high degree and produce a light-dark flop. These have proven to be particularly preferred for use in the coloring agent.

Suitable pigments based on a lamellar substrate platelet include, for example, the pigments of the VISIONAIRE series by Eckart.

Pigments based on a lenticular small substrate plate are available, for example, under the name Alegrace® Gorgeous from the company Schlenk Metallic Pigments GmbH.

Pigments based on a substrate platelet, which comprises a vacuum-metalized pigment, are available, for example, under the name Alegrace® Marvelous or Alegrace® Aurous from the company Schlenk Metallic Pigments GmbH.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ from 1.0 to 50 μm, preferably from 5.0 to 45 μm, preferably from 10 to 40 μm, in particular from 14 to 30 μm. The mean particle size D₅₀ can be determined, for example, using dynamic light scattering (DLS).

The pigments (f2) are preferably used in specific quantity ranges in the coloring agent (F). The coloring agent used in the method according to the invention for coloring may contain one or more pigments, for example in a total amount of from 0.01 to 10.0 wt. %, preferably from 0.1 to 5.0 wt. %, more preferably from 0.2 to 2.5 wt. % and very particularly preferably from 0.25 to 1.5 wt. %. Here, the indicated amounts relate to the total amount of all pigments used, which is set in relation to the total weight of the coloring agent.

In another very particularly preferred embodiment, a method according to the invention is characterized in that the coloring agent (F) contains—based on the total weight of the coloring agent (F)—one or more pigments (f2) in a total amount of 0.01 to 10.0 wt. %, preferably 0.1 to 5.0 wt. %, more preferably 0.2 to 2.5 wt. % and very particularly preferably 0.25 to 1.5 wt. %.

### Water Content in the Coloring Agent (F)

The coloring agent (F) described above is a ready-to-use agent which is applied to the keratin fibers. This ready-to-use agent preferably has a low to medium water content. It has been found that particularly those coloring agents are well-suited which contain—based on the total weight of the agent—0.1 to 50.0 wt. %, preferably 0.5 to 35.0 wt. %, more preferably 1.0 to 20.0 wt. %, and particularly preferably 1.5 to 15.0 wt. % water.

In another explicitly very particularly preferred embodiment, a method is characterized in that the coloring agent (F) contains—based on the total weight of the coloring agent (F)—0.1 to 50.0 wt. %, preferably 0.5 to 35.0 wt. %, more preferably 1.0 to 20.0 wt. % and particularly preferably 1.5 to 15.0 wt. % water.

### Cosmetic Carrier of Coloring Agent (F)

Due to the previously described water content of the coloring agent, which is preferably in the medium to low range, the main component of the cosmetic carrier in which the components (f1) and (f2) of the coloring agent are present is preferably non-aqueous. The cosmetic carrier is preferably a solvent and/or a polyethylene glycol.

Suitable solvents that can be used are, for example, solvents from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol. The use of 1,2-propylene glycol is very particularly preferred.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9 and PEG-10.

Another well-suited polyethylene glycol is PEG-32, for example. PEG-32 comprises 32 ethylene glycol units (x1=32), has a mean molar mass of 1500 g/mol and bears the CAS number 25322-68-3. PEG-32 is alternatively also referred to as PEG 1500 and can, for example, be purchased commercially from Clariant.

A very particularly well-suited polyethylene glycol with a medium molecular weight is for example PEG 6000, which can be obtained commercially from National Starch (China). The molecular weight of PEG 6000 is 6000 to 7500 g/mol, corresponding to an x3 value of 136 to 171.

### Shampoo (S)

In step (2) of the method according to the invention, a shampoo (S) is applied to the keratin fibers, in particular the human hair, which contains at least one amino-functionalized silicone polymer (s1).

Suitable, preferred and particularly preferred amino-functionalized silicone polymers (s1) are those substances and commercial products which have already been disclosed in detail in the description of the amino-functionalized silicone polymers (f1).

In the context of a further particularly preferred embodiment, a method according to the invention is characterized in that the shampoo (S) contains at least one amino-functionalized silicone polymer (s1) which comprises at least one structural unit of formula (Si-amino),

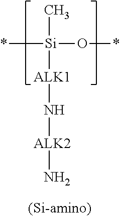

where
ALK1 and ALK2 represent, independently of one another, a linear or branched, divalent C₁-C₂₀ alkylene group.

In the context of a further particularly preferred embodiment, a method according to the invention is characterized in that the shampoo (S) contains at least one amino-functionalized silicone polymer (s1) which comprises structural units of formula (Si-I) and of formula (Si-II)

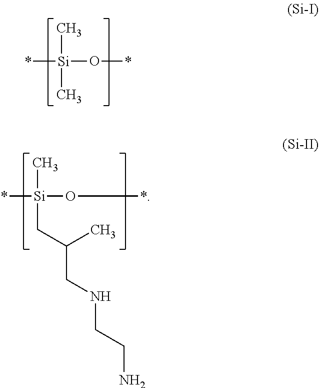

The commercial product DC 2-8566 or Dowsil 2-8566 Amino Fluid, which is sold commercially by the Dow Chemical Company and bears the name “Siloxanes and Silicones, 3-[(2-aminoethyl)amino]-2-methylpropyl Me, Di-Me-Siloxane” and the CAS number 106842-44-8, is particularly suitable for use in the shampoo (S). A further particularly preferred commercial product is Dowsil AP-8568 Amino Fluid, which is likewise sold commercially by Dow Chemical Company.

The shampoo (S) used in step (2) of the method may contain one or more amino-functionalized silicone polymers (s1), for example in a total amount of from 0.01 to 20 wt. %, preferably from 0.05 to 10 wt. %, more preferably from 0.1 to 5 wt. %, even more preferably from 0.2 to 3.5 wt. %, and very particularly preferably from 0.3 to 1.5 wt. %. Here, the indicated amounts relate to the total amount of all aminosilicones used, which is set in relation to the total weight of the shampoo.

In the context of another particularly preferred embodiment, a method according to the present invention is characterized in that the shampoo (S)—based on the total weight of the shampoo (S)—contains one or more amino-functionalized silicone polymers (s1) in a total amount of 0.01 to 20 wt. %, preferably from 0.05 to 10 wt. %, more preferably from 0.1 to 5 wt. %, still more preferably from 0.2 to 3.5 wt. %, and very particularly preferably from 0.3 to 1.5 wt. %.

A particularly good improvement in wash fastness was observed when the same amino-functionalized silicone polymer was used in the coloring agent (F) and in the shampoo (S). In this case, (f1) and (s1) are aminosilanes of the same structure or the same commercial products.

In the context of a further particularly preferred embodiment, a method according to the invention is characterized in that the coloring agent (F) and the shampoo (S) contain the same amino-functionalized silicone polymer (f1) and (s1), respectively.

### Coloring Agent (F) and Shampoo (S)

In the context of the method according to the invention, a coloring agent (F) and a shampoo (S) are applied to the keratin fibers in steps (1) and (2), wherein the coloring agent (F) and the shampoo (S) are different formulations.

The coloring agent (F) and shampoo (S) are used for different purposes and also contain different ingredients due to their different intended use. The coloring agent (F) is intended to achieve the coloring of the keratin fibers, so that the coloring agent contains the pigments (f2) responsible for the coloring.

The shampoo (S) is used for the subsequent cleaning of the keratin fibers and therefore contains the ingredients customary for cleaning, such as surfactants in particular.

For this reason, a coloring agent (F) according to the invention is preferably characterized in that it either contains no surfactants or contains them in smaller total amounts than the shampoo (S).

The shampoo (S) according to the invention is preferably characterized in that it does not contain any pigments.

The water content of the coloring agent and the shampoo is also preferably different.

### Water Content in the Shampoo

The shampoo (S) contains the amino-functionalized silicone polymer(s) (s1) particularly preferably in an aqueous cosmetic carrier. It has been found to be preferred if the water content in the shampoo (S)—based on the total weight of the shampoo (S)—is in the region of from 50 to 99 wt. %, preferably from 60 to 97 wt. %, more preferably from 70 to 95 wt. %, and particularly preferably from 75 to 93 wt. %.

In a further preferred embodiment, a method according to the invention is therefore characterized in that the shampoo (S) contains—based on the total weight of the shampoo (S)—from 50 to 99 wt. %, preferably from 60 to 97 wt. %, more preferably from 70 to 95 wt. %, and particularly preferably from 75 to 93 wt. %, water.

### Surfactants in the Shampoo (S)

The shampoo (S) is applied by the user or hairdresser in particular when the previously colored keratin fibers are to be cleaned. The work carried out in the context of this application has shown that the color loss associated with the application of the shampoo (S) could be reduced or minimized by the presence of at least one aminosilicone (s1) in the shampoo. For the purpose of cleaning, the shampoo particularly preferably additionally contains at least one surfactant from the group of anionic, amphoteric, zwitterionic, cationic and/or nonionic surfactants.

In a further preferred embodiment, a method according to the invention is therefore characterized in that the shampoo (S) contains at least one anionic, amphoteric, zwitterionic, cationic and/or nonionic surfactant.

The term surfactants (T) is understood to mean interface-active substances which form adsorption layers on upper and boundary surfaces or can aggregate in volume phases to form micelle colloids or lyotropic mesophases. A distinction is made between anionic surfactants consisting of a hydrophobic functional group and a negatively charged hydrophilic head group, amphoteric surfactants which bear both a negative and a compensating positive charge, cationic surfactants which have a positively charged hydrophilic group in addition to a hydrophobic functional group, and non-ionic surfactants which have no charges but strong dipole moments and are strongly hydrated in aqueous solution.

Cationic surfactants are understood to mean surfactants, i.e. surface-active compounds, each having one or more positive charges. Cationic surfactants contain exclusively positive charges. Typically, these surfactants are composed of a hydrophobic part and a hydrophilic head group, with the hydrophobic part generally consisting of a hydrocarbon framework (e. g., consisting of one or two linear or branched alkyl chains), and the positive charge(s) being located in the hydrophilic head group. Examples of cationic surfactants are:
quaternary ammonium compounds which, as hydrophobic functional groups, can bear one or two alkyl chains having a chain length of 8 to 28 C atoms,
quaternary phosphonium salts substituted with one or more alkyl chains having a chain length of 8 to 28 C atoms, or
tertiary sulfonium salts.

Furthermore, the cationic charge can also be part of a heterocyclic ring (for example an imidazolium ring or a pyridinium ring) in the form of an onium structure. In addition to the functional unit carrying the cationic charge, the cationic surfactant can also contain further uncharged functional groups, as is the case, for example, with esterquats. The cationic surfactants are used in a total amount from 0.1 to 45 wt. %, preferably 1 to 30 wt. %, and very particularly preferably from 1 to 15 wt. %, based on the total weight of each agent.

Non-ionic surfactants contain, for example as a hydrophilic group, a polyol group, a polyalkylene glycol ether group or a combination of polyol and polyglycol ether. Such compounds include, for example
addition products of 2 to 50 mol ethylene oxide and/or 0 to 5 mol propylene oxide to linear and branched fatty alcohols having 6 to 30 C atoms, the fatty alcohol polyglycol ethers or the fatty alcohol polypropylene glycol ethers or mixed fatty alcohol polyethers,
addition products of 2 to 50 mol ethylene oxide and/or 0 to 5 mol propylene oxide to linear and branched fatty alcohols having 6 to 30 C atoms, the fatty acid polyglycol ethers or the fatty acid polypropylene glycol ethers or mixed fatty alcohol polyethers,
addition products of 2 to 50 mol ethylene oxide and/or 0 to 5 mol propylene oxide to linear and branched alkyl phenols having 8 to 15 C atoms in the alkyl group, the alkyl phenol polyglycol ethers or the alkyl polypropylene glycol ethers or mixed alkyl phenol polyethers,
addition products end-capped with a methyl- or C2-C6 alkyl functional group of 2 to 50 mol ethylene oxide alkyl and/or 0 to 5 mol propylene oxide to linear and branched fatty alcohols with 8 to 30 carbon atoms, to fatty acids with 8 to 30 carbon atoms, and to alkylphenols with 8 to 15 carbon atoms in the alkyl group, such as those available type under the trade names Dehydol® LS, Dehydol® LT (Cognis),
C12-C30 fatty acid mono- and diesters of products of the addition of 1 to 30 mol of ethylene oxide to glycerol,
addition products of 5 to 60 mol ethylene oxide to castor oil and hydrogenated castor oil,
polyol fatty acid esters, such as the commercial product Hydagen® HSP (Cognis) or Sovermol® types (Cognis),
alkoxylated triglycerides,
alkoxylated fatty acid alkyl esters of the formula (Tnio-1)

R¹CO—(OCH₂CHR²)_{w}OR³ (Tnio-1)

wherein R¹CO is a linear or branched, saturated and/or unsaturated acyl functional group having 6 to 22 carbon atoms, R² is hydrogen or methyl, R³ is linear or branched alkyl functional groups having 1 to 4 carbon atoms and w is a number from 1 to 20,
amine oxides,
hydroxy mixed ethers, as are described, for example, in DE-OS 19738866,
sorbitan fatty acid esters and addition products of ethylene oxide to sorbitan fatty acid esters such as the polysorbates,
sugar fatty acid esters and addition products of ethylene oxide to sugar fatty acid esters,
addition products of ethylene oxide to fatty acid alkanolamides and fatty amines,
sugar surfactants of the type of the alkyl and alkenyl oligoglycosides according to formula (E4-II),

R⁴O-[G]ₚ (Tnio-2)

in which R⁴ represents an alkyl group or alkenyl functional group having 4 to 22 carbon atoms, G represents a sugar functional group having 5 or 6 carbon atoms, and p represents numbers from 1 to 10. They can be obtained in accordance with the pertinent methods of preparative organic chemistry. The alkyl and alkenyl oligoglycosides can be derived from aldoses or ketoses having 5 or 6 carbon atoms, preferably from glucose. The preferred alkyl and/or alkenyl oligoglycosides are therefore alky and/or alkenyl oligoglucosides. The index number p in the general formula (Tnio-2) indicates the degree of oligomerization (DP), i.e., the distribution of mono- and oligoglycosides, and represents a number between 1 and 10. While p must always be an integer in the individual molecular and can especially assume the values p=1 to 6 here, for a certain alkyl oligoglycoside the value p is an analytically determined calculated quantity, which is usually a fraction. Alkyl and/or alkenyl oligoglycosides having an average degree of oligomerization p of 1.1 to 3.0 are preferably used. From the perspective of application technology, alkyl and/or alkenyl oligoglycosides whose degree of oligomerization is less than 1.7 and lies in particular between 1.2 and 1.4 are preferred. The alkyl group or alkenyl group R4 can be derived from primary alcohols having 4 to 11, preferably 8 to 10, carbon atoms. Typical examples include butanol, caproic alcohol, caprylic alcohol, capric alcohol and undecyl alcohol as well as the industrial mixtures thereof, as obtained for example by the hydrogenation of industrial fatty acid methyl esters or in the course of the hydrogenation of aldehydes in the Roelen oxosynthesis reaction. Alkyl oliglucosides with a C₈-C₁₀ chain length (DP=1 to 3) which accumulate as the first runnings in the separation, by means of distillation, of industrial C₈-C₁₈ coco fatty alcohol and which may be contaminated with a C₁₂ alcohol content of less than 6 wt. % and alkyl oligoglucosides based on industrial C_{9/11} oxo alcohols (DP=1 to 3) are preferred. The alkyl or alkenyl functional group R₁₅ can furthermore also be derived from primary alcohols having 12 to 22, preferably 12 to 14 carbon atoms. Typical examples include lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and the industrial mixtures thereof, which can be obtained as described above. Alkyl oligoglucosides based on hardened C_{12/14} coconut alcohol and having a DP of 1 to 3 are preferred.
Sugar surfactants of the fatty-acid-N-alkylpolyhydroxyalkyl amide type, a non-ionic surfactant of formula (Tnio-3),

R⁵CO—NR⁶—[Z] (Tnio-3)

in which R¹CO represents an aliphatic acyl functional group having 6 to 22 carbon atoms, R⁶ represents hydrogen, an alkyl or hydroxy alkyl functional group having 1 to 4 carbon atoms and [Z] represents a linear or branched polyhydroxyalkyl functional group having 3 to 12 carbon atoms and 3 to 10 hydroxyl groups. Fatty acid-N-alkylpolyhydroxyalkyl amides are known substances which can be conventionally obtained by the reductive amination of a reducing sugar with ammonia, an alkylamine or an alkanolamine and subsequent acylation with a fatty acid, a fatty acid alkyl ester or a fatty acid chloride. Fatty acid-N-alkylpolyhydroxyalkyl amides are preferably derived from reducing sugars, glucose in particular, having 5 or 6 carbon atoms. The preferred fatty acid-N-alkyl-polyhydroxyalkyl amides are therefore fatty acid-N-alkyl glucamides represented by formula (Tnio-4):

R⁷CO—(NR⁸)—CH₂—[CH(OH)]₄—CH₂OH (Tnio-4)

The use of glucamides of formula (Tnio-4), in which R′ represents hydrogen or an akyl group and R⁷CO represents the acyl functional group of caproic acid, caprylic acid, capric acid, lauric acid, myristic acid, palmitic acid, palm oleic acid, stearic acid, isostearic acid, oleic acid, elaidic acid, petroselic acid, linoleic acid, linolenic acid, arachidic acid, gadoleic acid, behenic acid or erucic acid or industrial mixtures thereof is preferred. Fatty acid-N-alkyl glucamides of formula (Tnio-4) obtained by reductive amination of glucose with methylamine and subsequent acylation using lauric acid or C_{12/14} coconut fatty acid or an appropriate derivative thereof are particularly preferred. Furthermore, the polyhydroxyalkyl amides may also be derived from maltose and palatinose.

Further typical examples of non-ionic surfactants are fatty acid amide polyglycol ethers, fatty amine polyglycol ethers, mixed ethers or mixed formals, protein hydrolyzates (in particular wheat-based vegetable products) and polysorbates.

Addition products of alkylene oxide to saturated linear fatty alcohols and fatty acids containing from 2 to 30 mol ethylene oxide per mol of fatty alcohol or acid, and sugar surfactants, have proved to be suitable as preferred non-ionic surfactants. Preparations with excellent properties are likewise obtained if they contain fatty acid esters of ethoxylated glycerol as the nonionic surfactants.

Furthermore, the shampoos (S) according to the invention can also contain at least one anionic surfactant. Anionic surfactants are surface-active agents with exclusively anionic charges (neutralized by a corresponding counter cation). Examples of anionic surfactants are fatty acids, alkyl sulfates, alkyl ether sulfates, and ether carboxylic acids having 12 to 20 C atoms in the alkyl group and up to 16 glycol ether groups in the molecule.

The surfactants described above are preferably used in the suitable amount ranges in the shampoo (S). Thus, the shampoo (S) may contain—based on the total weight of the shampoo (S)—one or more surfactants in a total amount of from 0.1 to 20 wt. %, preferably from 0.2 to 15 wt. % and particularly preferably from 0.3 to 10 wt. %.

Further Optional Ingredients in the Coloring Agent (F) and/or in the Shampoo (S)

In addition to the already described components essential to the invention, the coloring agent and/or the shampoo can also contain other optional ingredients, such as solvents, anionic, non-ionic, zwitterionic and/or cationic polymers; structurants such as glucose, maleic acid and lactic acid, hair-conditioning compounds such as phospholipids, for example lecithin and cephalins; perfume oils, dimethyl isosorbide and cyclodextrins; fiber structure-improving agents, in particular mono-, di- and oligosaccharides, for example glucose, galactose, fructose and lactose; dyes for coloring the product; anti-dandruff active ingredients, such as piroctone olamine, zinc omadine and climbazole; amino acids and oligopeptides; animal and/or vegetable-based protein hydrolyzates, as well as in the form of their fatty acid condensation products or optionally anionically or cationically modified derivatives; vegetable oils; light stabilizers and UV blockers; active ingredients, such as panthenol, pantothenic acid, pantolactone, allantoin, pyrrolidinone carboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes, such as fatty alcohols, beeswax, montan wax and kerosenes; swelling and penetrating agents, such as glycerol, propylene glycol monoethyl ether, carbonates, hydrogen carbonates, guanidines, ureas and primary, secondary and tertiary phosphates; opacifiers, such as latex, styrene/PVP and styrene/acrylamide copolymers; pearlescent agents, such as ethylene glycol mono- and distearate as well as PEG-3-distearate; and propellants such as propane-butane mixtures, N₂O, dimethyl ether, CO₂ and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according the invention preferably always in amounts of 0.0001 to 25 wt. %, in particular of 0.0005 to 15 wt. %, relative to the total weight of the particular agent.

### Steps (1) and (2) of the Method

The method according to the invention comprises the application of a coloring agent (F) to the keratin fibers in step (1) and the application of a shampoo (S) to the keratin fibers in step (2).

As already described above, it is particularly preferred if the keratin fibers are first colored by applying the coloring agent (F), and then the shampoo (S) is applied. The time at which the shampoo (S) is applied depends on the needs of the user and can be adapted to their habits.

For example, it is possible to apply the shampoo (S) to the freshly colored, still wet or damp keratin fibers so that there is a period of only a few minutes to a few hours between rinsing out the coloring agent (F) and applying the shampoo (S).

However, it is particularly preferable to apply the shampoo (S) at the time when the user usually wants to wash their keratin fibers or hair for the first time after coloring. This time is usually between 1 to 3 days after coloring.

In a further embodiment, a method is very particularly preferred which comprises
(1) applying the coloring agent (F) to the keratin fibers in a first step, then
(2) applying the shampoo (S) to the keratin fibers in a second step, wherein between steps (1) and (2) there is a period of maximum 72 hours, preferably maximum 48 hours, particularly preferably maximum 24 hours.

The application of the shampoos can also be repeated one or more times.

In a further embodiment, a method is very particularly preferred which comprises
(1) applying the coloring agent (F) to the keratin fibers in a first step, then
(2) applying the shampoo (S) to the keratin fibers in a second step, wherein between steps (1) and (2) there is a period of maximum 72 hours, preferably maximum 48 hours, particularly preferably maximum 24 hours, wherein step (2) is repeated one or more times.

### Multi-Component Packaging Unit

To increase convenience of use, the user is preferably provided with all the required agents in the form of a multi-component packaging unit (kit-of-parts).

The present invention therefore thirdly relates to a multi-component packaging unit (kit-of-parts) for coloring, or for coloring and washing, keratin fibers, in particular human hair, comprising, packaged separately from one another:
a first container having a first partial formulation of the coloring agent (F), which partial formulation contains
(f1) at least one amino-functionalized silicone polymer, and
a second container having a second partial formulation of the coloring agent (F), which partial formulation contains
(f2) at least one pigment, and
a third container having a shampoo (S) which contains
(s1) at least one amino-functionalized silicone polymer,
wherein the coloring agent (F), the shampoo (S) and the components (f1), (f2) and (s1) have already been disclosed in detail in the description of the first subject matter of the invention.

The multi-component packaging unit (kit-of-parts) can also comprise one or more further containers having further formulations.

Preferred is also a multi-component packaging unit (kit-of-parts) for coloring and washing keratin fibers, in particular human hair, comprising, packaged separately from one another:
a first container having a first partial formulation of the coloring agent (F), which partial formulation contains
(f1) at least one amino-functionalized silicone polymer, and
a second container having a second partial formulation of the coloring agent (F), which partial formulation contains
(f2) at least a first pigment, and
a third container having a third partial formulation of the coloring agent (F), which partial formulation contains
(f2) at least a second pigment, and
a fourth container having a shampoo (S) which contains
(s1) at least one amino-functionalized silicone polymer,
wherein the coloring agent (F), the shampoo (S) and the components (f1), (f2) and (s1) have already been disclosed in detail in the description of the first subject matter of the invention.

The statements made about the method according to the invention apply mutatis mutandis in regard to the other preferred embodiments of the multi-component packaging unit according to the invention.

### Improvement in Color Retention

For the purposes of the present invention, an improvement in color retention is understood in particular to mean an improvement in wash fastness, i.e. when the method according to the invention is applied, fewer pigments are removed from the hair in subsequent hair washes. The color retention can be quantified, for example, by colorimetric measurements (measurement of the L, a, b values) and calculation of the color difference. The smaller the color difference between the washed and unwashed hair, the better the wash fastness or color retention.

A third subject matter or the present application relates to the use of a shampoo (S) containing at least one amino-functionalized silicone polymer (s1) for improving color retention on keratin fibers, in particular human hair, which have been colored by application of a coloring agent (F) containing at least one amino-functionalized silicone polymer (f1) and at least one pigment (f2),
wherein the coloring agent (F), the shampoo (S) and the components (f1), (f2) and (s1) have already been disclosed in detail in the description of the first subject matter of the invention.

The statements made about the method according to the invention apply mutatis mutandis in regard to the other preferred embodiments of the use according to the invention.

### Examples

### 1. Formulations

The following formulations were produced (all information is in wt. % unless indicated otherwise)

| Ready-to-use coloring agent (F) | F1 | F2 |
|---|---|---|
| DOWSIL AP-8568 Amino Fluid (aminosilicone (f1)) | 0.75 | 0.75 |
| Unipure Red LC 3079 (pigment (f2)) | 0.38 | 0.38 |
| VPM aluminum pigment (D(50) = 12 microns), | — | 2.50 |
| (Alegrace Marvelous D 12/77-1 Shiny Silver) | | |
| (pigment (f2)) 10% aqueous dispersion | | |
| Water | 10.0 | 10.0 |
| PPG-3 benzyl ether myristate | 1.5 | 1.5 |
| Ucon Fluid AP (PPG-14 butyl ether) | 1.0 | 1.0 |
| Phenoxyethanol | 0.2 | 0.2 |
| 4-hydroxyacetophenone | 0.2 | 0.2 |
| Polyethylene glycol (molecular weight 6000 g/mol) | 10.0 | 10.0 |
| Polyethylene glycol (molecular weight 400 g/mol) | up | up |
| | to 100 | to 100 |

| Shampoo (S) | (S1) | (S2) |
|---|---|---|
| Sodium laureth sulfate (25% aqueous solution) | 30.00 | 30.00 |
| Cocoamidopropyl betaine (37% aqueous solution) | 4.00 | 4.00 |
| Sodium lauryl sulfate | — | 1.00 |
| Ethylene glycol distearate | 0.50 | 0.50 |
| PEG-100 stearate | 0.50 | — |
| Dimethicone 5 cSt | 0.50 | — |
| DOWSIL AP-8568 Amino Fluid (aminosilicone | 1.00 | 1.25 |
| (s1)) | | |
| Trideceth-10 | 0.50 | — |
| Trideceth-5 | 0.25 | |
| Sodium chloride | 1.00 | 1.00 |
| PPG-5-CETETH-20 | 0.50 | 0.50 |
| Fumaric acid | 0.40 | 0.40 |
| Sodium hydroxide (50% aqueous solution) | 0.20 | 0.20 |
| Guar hydroxypropyltrimonium chloride | 0.20 | 0.20 |
| Cetrimonium chloride | 1.00 | 1.0 |
| Phenoxyethanol | 0.50 | — |
| Sodium benzoate | 0.50 | 0.50 |
| Citric acid | 0.10 | 0.10 |
| Water | up to 100 | up to 100 |

What is claimed is:

1. A method for coloring keratin fibers, in particular human hair, comprising the steps of:
(1) applying a coloring agent (F) to the keratin fibers, said coloring agent (F) comprising
(f1) at least one amino-functionalized silicone polymer, and
(f2) at least one pigment, and
(2) applying a shampoo agent (S) to the keratin fibers, said shampoo (S) comprising
(s1) at least one amino-functionalized silicone polymer, and
wherein the coloring agent (F) and the shampoo (S) are different formulations.

2. The method according to claim 1, wherein the coloring agent (F) contains at least one amino-functionalized silicone polymer (f1) which comprises at least one structural unit of the formula (Si-amino), 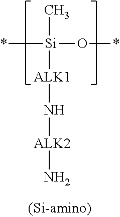
where
ALK1 and ALK2 represent, independently of one another, a linear or branched divalent C₁-C₂₀ alkylene group.

3. The method according to claim 1, wherein the coloring agent (F) contains at least one amino-functionalized silicone polymer (f1) comprising structural units of the formula (Si-I) and of the formula (Si-II)

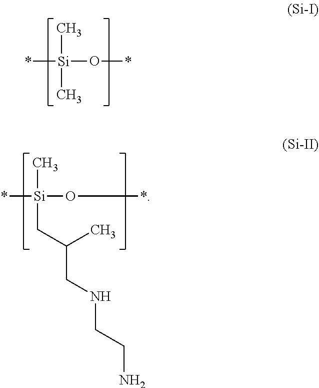

4. The method according to claim 1, wherein the coloring agent (F)—based on the total weight of the coloring agent (F)—comprises one or more amino-functionalized silicone polymers (f1) in a total amount of 0.1 to 20 wt. %.

5. The method according to claim 1, wherein the coloring agent (F)—based on the total weight of the coloring agent (F)—comprises one or more amino-functionalized silicone polymers (f1) in a total amount of 0.2 to 10 wt. %.

6. The method according to claim 1, wherein the coloring agent (F)—based on the total weight of the coloring agent (F)—comprises one or more amino-functionalized silicone polymers (f1) in a total amount of 0.5 to 2.0 wt. %.

7. The method according to claim 1, wherein the coloring agent (F) contains at least one inorganic pigment (f2), wherein the at least one inorganic pigment is selected from the group consisting of: colored metal oxides, metal hydroxides, metal oxide hydrates, silicates, metal sulfides, complex metal cyanides, metal sulphates, and bronze pigments and/or from mica-based colored pigments which are coated with at least one metal oxide and/or metal oxychloride.

8. The method according to claim 1, wherein the coloring agent (F) contains at least one organic pigment (f2), wherein the organic pigment is selected from the group consisting of carmine, quinacridone, phthalocyanine, sorghum, blue pigments having the Color Index Numbers Cl 42090, CI 69800, CI 69825, CI 73000, CI 74100, CI 74160, yellow pigments having the Color Index Numbers CI 11680, CI 11710, CI 15985, CI 19140, CI 20040, CI 21100, CI 21108, CI 47000, CI 47005, green pigments having the Color Index Numbers CI 61565, CI 61570, CI 74260, orange pigments having the Color Index Numbers CI 11725, CI 15510, CI 45370, CI 71105, red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and CI 75470.

9. The method according to claim 1, wherein the coloring agent (F) comprises at least one pigment (f2) selected from the group consisting of: pigments based on a lamellar substrate platelet, pigments based on a lenticular substrate platelet, and vacuum-metallized pigments.

10. The method according to claim 1, wherein the coloring agent (F) contains, based on the total weight of the coloring agent (F), from 0.1 to 50.0 wt. % water.

11. The method according to claim 1, wherein the coloring agent (F) contains, based on the total weight of the coloring agent (F), from 1.5 to 15.0 wt. %, water.

12. The method according to claim 1, wherein the shampoo (S) comprises at least one amino-functionalized silicone polymer (s1) comprising at least one structural unit of formula (Si-amino), 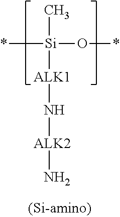
where
ALK1 and ALK2 represent, independently of one another, a linear or branched, divalent C₁-C₂₀ alkylene group.

13. The method according to claim 1, wherein the shampoo (S) comprises at least one amino-functionalized silicone polymer (s1) comprising structural units of the formula (Si-I) and of the formula (Si-II)

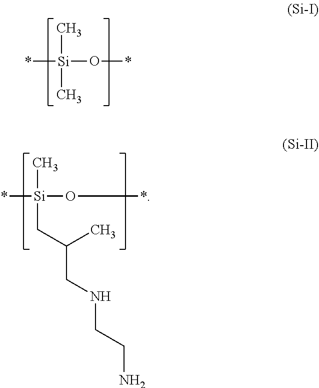

14. The method according to claim 1, wherein the shampoo (S)—based on the total weight of the shampoo (S)—comprises one or more amino-functionalized silicone polymers (s1) in a total amount of 0.01 to 20 wt. %.

15. The method according to claim 1, wherein the shampoo (S)—based on the total weight of the shampoo (S)—comprises one or more amino-functionalized silicone polymers (s1) in a total amount of 0.3 to 1.5 wt. %.

16. The method according to claim 1, wherein the coloring agent (F) and the shampoo (S) contain the same amino-functionalized silicone polymer (f1) and (s1), respectively.

17. The method according to claim 1, wherein the shampoo (S) contains—based on the total weight of the shampoo (S)—from 50 to 99 wt. % water.

18. The method according to claim 1, wherein the shampoo (S) contains at least one anionic, amphoteric, zwitterionic, cationic and/or nonionic surfactant.

19. The method according to claim 1, wherein there is a period of a maximum of 72 hours between applying the coloring agent (F) to the keratin fibers and applying the shampoo (S) to the keratin fibers.

20. A multi-component packaging unit (kit-of-parts) for coloring and washing keratin fibers, in particular human hair, comprising, packaged separately from one another:
a first container having a first partial formulation of the coloring agent (F), which partial formulation contains
(f1) at least one amino-functionalized silicone polymer, and
a second container having a second partial formulation of the coloring agent (F), which partial formulation contains
(f2) at least one pigment, and
a third container having a shampoo (S) which contains
(s1) at least one amino-functionalized silicone polymer.
